(19) United States
(12) Patent Application Publication
(10) Pub. No.: US 2026/0345202 A1
(43) Pub. Date: Nov. 5, 2026

(54) COMB FOR THE TREATMENT OF PEDICULOSIS

(71) Applicants: Martina Maria RAY, Dique Lujan, Buenos Aires (AR); LACUNA S.A., Montevideo (UY)

(72) Inventor: Juan Martin SANZ, Ciudad Autonoma de Buenos Aires (AR)

(73) Assignees: Martina Maria RAY, Dique Lujan, Buenos Aires (AR); LACUNA S.A., Montevideo (UY)

(21) Appl. No.: 19/119,038

(22) PCT Filed: Sep. 18, 2023
(86) PCT No.: PCT/EP2023/075559

§ 371 Date: Apr. 7, 2025

(30) Foreign Application Priority Data
Oct. 26, 2022 (AR) P20220102907

(51) Int. Cl. A61B 17/50; A61B 17/00

(52) U.S. Cl. CPC A61B 17/50; A61B 2017/00526; A61B 2017/00747; A61B 2017/505

(57) ABSTRACT

A comb for the treatment of pediculosis which provides an innovative arrangement of the grooves of the teeth in order to significantly improve the efficiency in the removal of nits and/or lice without reducing combability.

### BACKGROUND OF THE INVENTION

### Field of the Invention

The present invention relates to hair cleaning and more particularly to the devices used for the treatment of pediculosis, in order to remove the nits or eggs of the lice that cling to the hair, and even more particularly the invention relates to a new comb for the treatment of pediculosis with an arrangement and differentiation of the grooves in order to improve the effectiveness in the removal of lice eggs (nits) and lice from the hair without diminishing the effectiveness of combing or combability.

### Description of the Prior Art

As is well known in the art, pediculosis is a common problem that affects countless individuals, and more particularly represents an obstacle in the student population since school-age children acquire it more easily when they are in contact with their classmates in the classroom or educational institution they attend. Likewise, contagion between children not only occurs in the educational field but also in squares, houses and places where two or more people meet, provided that at least one is a possible carrier of nits and/or lice.

When a carrier person is in contact with a non-carrier one and contagion occurs, the lice settle in the hair and implant the well-known nits there, that is, the eggs of Pedeculus humanus capitis and similar species, more commonly called lice.

Many chemical compounds have been developed for the treatment of this parasitism, as well as countless combs that aim to treat this problem by removing lice and nits, thus avoiding the application of chemical products that cause side effects. There are thus various designs of combs that comprise a handle to which a plurality of teeth or steel wires combs are attached, wherein the separation between teeth is studied so that only a hair passes tightly between adjacent teeth so that any adherence, particularly a nit, is retained between the teeth of the comb. By passing the comb through the entire hair of the patient, it is possible to remove as many nits as possible.

One of the combs that has most effectively achieved the objective of removing nits with the greatest success is the one disclosed in U.S. Pat. No. 5,873,374, which describe, as more specifically defined in their Claims, a cleaning comb with needles having a roughness on their peripheral surface comprising a handle and a plurality of teeth, wires or needles, preferably made of steel, each needle having an end for gripping a handle, a leading end and a peripheral surface, the leading end of each needle having a sharp rounded point. In accordance with the concepts of these patents, the novelty of said prior art comb consists in that the peripheral surface of each needle is provided with a roughness intended to catch and drag the nit attached to the hair. The comb also has other characteristics such as that the distance between a needle and the neighboring needle is between 50 μm and 100 μm, each needle having a total length in the range of 40 to 80 mm and a useful length outside the handle, in the range from 20 to 60 mm.

The roughness characteristic referred to in the above cited US Patent is preferably based on the provision of helical channels, which have, for example, a pitch of less than 4 mm. This roughness can also be made up of circumferential channels that are separated from each other by a distance of between 0.5 and 3 mm, and said channels can have a depth of less than 0.2 mm.

Said patented comb has other characteristics such as that it is made of steel, the number of needles is between 10 and 100 needles, more precisely 33 needles, and that the leading edges of the needles together form a curved edge. In addition, the prongs or needles have their proximal ends retained in the handle and their distal ends, which all end aligned on said leading edge, must be delicately finished in a tapered, refined and rounded shape, so that combability is not diminished and that, at the same time, allows the hair to enter the space between the teeth or needles.

Another type of comb may be that disclosed in Publication US 2002/0078972 A1 by Levandosvky. According to FIG. 1 related to the prior art which corresponds to FIG. 4 of Levandosvky and which is cited herein as a reference, a comb 1 is taught to clean the hair of adhering matter, particularly lice and nits, the comb comprising a plurality of needles 3 defining a gap of between 5 microns to 100 microns therebetween. Each needle has at least one “U”-shaped material collecting channel 8 that extends along two combing sides 6 and 7 defined by the needles, as can be seen in FIG. 1 related to the prior art.

Each collecting channel 8 of each needle is arranged in the vicinity of the leading edge of the reed and lies side by side to form a general collecting channel or width. Each collecting channel 8 is defined by a recess 9 with right angles. On the other hand, Levandosvky teaches that each needle has a circular cross section, preferably a cylindrical shape, which defines a needle diameter of about 1 mm to about 1.5 mm. Thus, each recess 9, and therefore the channel 8, has a depth that is approximately 20% to 25% of the diameter of the needle and a width of approximately 3 mm to approximately 8 mm. Likewise, each needle 3 has a plurality of slots 13 spaced from one another at a distance of between 3 mm and 5 mm. Like the recesses 9, the slots 13 are provided on both sides 6 and 7 with combing.

The Levandosvky slots 13 are made by a cutting machine such as a grinder, which causes a substantial reduction of the cross section of the tooth or needle to at least half the diameter. Said reduction in diameter produces a significant weakening of the needle and leads to a loss of resistance to bending thereof. This forces Levandowsky to distance the slots 13 from one another because if they were very close the tooth or needle they would lose all the necessary resistance and would break under a slightly greater effort than usual. This is important because the aim is to have the slots as close to each other to improve the effectiveness of nit and louse removal and removal. Likewise, the use of a collecting area 8 is not useful since the nits will inevitably die when they are detached, being a space that could be used for other purposes.

On the other hand, it can be seen that the arrangement of the slots 13 is perpendicular to the needles. This loses the effect that the slots have to attack the nits, that is, if the comb is passed perpendicular to the hair, they would not work. Added to a very small number of slots, they make the Levandosvky comb unsuitable for the treatment of head lice. Doubling the channels on both leading sides only increases inefficiency due to loss of flexural strength of the teeth. This is because removing material from the teeth weakens them and decreases performance. The inertia with respect to the axis of work of the tooth is proportional to the diameter, so the removal of 20 to 25% of material decreases the resistance to bending and the effectiveness of the removal of lice and nits in the same proportion.

On the other hand, the Levandosvky channels 8 are the product of a grinding process, which is proposed with a depth “a” between 0.2 D to 0.25 D. In the case of a diameter of 1 mm to 1.5 mm this translates to 0.2 mm to 0.375 mm. This means that the channel, only in the best of cases, with a tooth diameter of 1 mm will increase the effective separation with respect to the adjacent channel by more than 100%, that is, from 0.10 to 0.22 mm. While, at the other end with a tooth of diameter 1.5 mm, the separation between contiguous teeth channels will be triple, that is to say, 0.3 mm. In summary, the spacing between teeth is not modified, but the effective between channels is affected since it increases between 100% and 200%. Likewise, the width of the channel from 3 to 8 mm decreases the number of channels necessary for the removal of nits. It is also important to note that Levandovsky seeks to collect in the slots, very wide and deep by the way, all the material, lice, nits, dirt, extracted from the hair without addressing the effectiveness in the removal of nits and lice.

By virtue of the current state of the art in relation to combs for the treatment of pediculosis, it would be very convenient to have a new comb that improves the effectiveness or efficiency in the removal of nits and/or lice for the treatment of pediculosis without affect the person's hair during the process of cleaning and combing.

### BRIEF DESCRIPTION OF THE INVENTION

It is therefore an object of the present invention to provide a new comb for the treatment of pediculosis comprising needles, teeth or wires capable of retaining, trapping and dragging nits, dirt and lice without diminishing combability, where said needles or teeth comprise a novel peripheral groove on only one side or face of the comb.

It is yet another object of the present invention to provide a comb for the treatment of pediculosis that, through said peripheral grooves, guarantees an effective removal of nits and foreign matter adhered to the hair, which easily enters the gaps between teeth whatever the way the comb is ran through the hair.

It is also another object of the present invention to provide a comb for the treatment of pediculosis in which the groove is made by laser on the periphery of the tooth and it does not reach the equator of the tooth cross-section, avoiding any type of reduction in diameter that may affect the efficacy of treatment and resistance of the teeth.

It is yet another object of the present invention to provide a comb for the treatment of pediculosis defined on one side where each needle or tooth is provided with a plurality of said peripheral grooves while the other side is smooth or does not contain grooves.

It is still another object of the present invention to provide a comb for the treatment of pediculosis in which the peripheral grooves are arranged discontinuously apart from each other and at such a distance as to guarantee the effective removal of nits. Arranged discontinuously means that a slot does not extend all around the needle or tooth but in a selected part of it.

It is also another object of the present invention to provide a comb for the treatment of pediculosis of the type that comprises a grip handle from which a plurality of teeth project, said teeth having a proximal end retained in said handle and a free distal end which together define a leading edge designed to first come into contact with a person's hair and to pass through the hair and allow the process of removing nits and/or lice, wherein each of said teeth comprises a roughness defined by a plurality of notches or grooves independent of each other and separated from each other, each of said grooves being made in a peripheral portion of each tooth so as to project or extend from a north pole of the tooth cross-section and circumferentially around the tooth and towards both sides up to the vicinity of the equator of the cross-section of the tooth.

### BRIEF DESCRIPTION OF THE DRAWINGS

For greater clarity and understanding of the object of the present invention, it has been illustrated in several figures, in which the invention has been represented in one of the preferred forms of embodiment, all by way of example, wherein:

FIG. 1 shows a partial side view of the comb for the treatment of pediculosis according to Publication US 2002/0078972 A1 and which document is incorporated by reference;

FIG. 2 shows a front view of the comb for the treatment of pediculosis according to the present invention;

FIG. 3 shows an enlarged view of a leading area of the comb for the treatment of pediculosis according to the present invention, where the arrangement of the grooves object of the invention can be observed in more detail;

FIG. 4 shows a section view of some teeth that make up the comb according to the present invention, where the peripheral development of the grooves object of the present invention can be observed in more detail, which do not reach the equator of the section in order to avoid being located in the space between the needles through which the hair slides;

FIG. 5 shows an enlarged and perspective view of a portion of the comb of the present invention, where the peripheral arrangement of the grooves according to the present invention can be observed;

FIG. 6 shows a schematic view of a part of the comb of the invention, where some teeth are illustrated which are close to removing the corresponding nits and/or lice; and

FIG. 7 shows an enlarged view and merely for illustrative purposes of the removal of nits and/or lice by using the comb of the present invention.

### DETAILED DESCRIPTION OF THE INVENTION

Referring now to the figures, it can be seen that the invention consists of a new comb for the treatment of pediculosis which offers a novel arrangement of the grooves of the teeth or needles in order to significantly improve the efficiency in the removal of nits and/or lice without diminishing combability. That is, the combability of the hair is not affected and the damage of the hair is acceptable if the hair can be cut when it finds a nit, since pulling the nit by pressure generates pain and goes against the cleaning process of the child's head. It is preferable to cut the hair with nits than to pull the nit to detach it from the hair. In general, the laser edge according to the present invention cuts the nit without pulling the hair or may in some cases also cut the hair where it was attached.

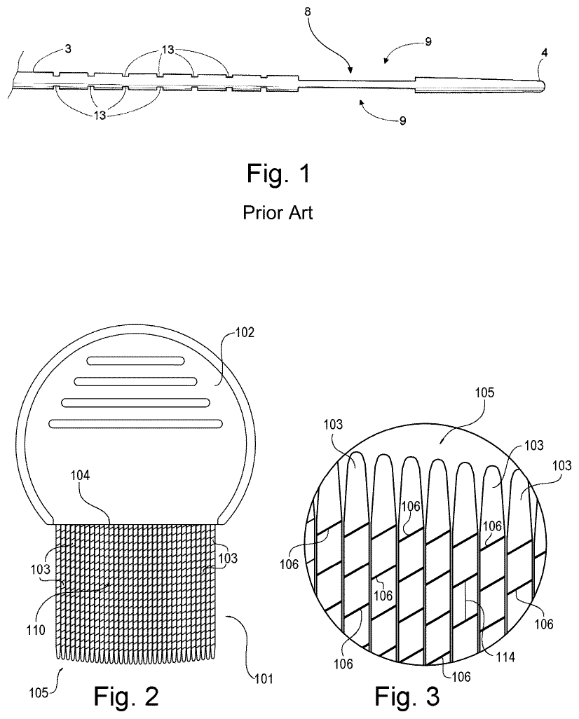

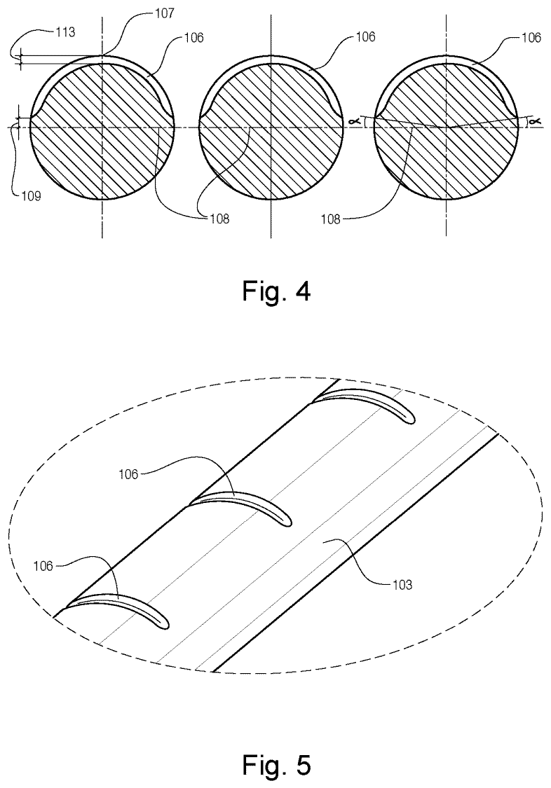

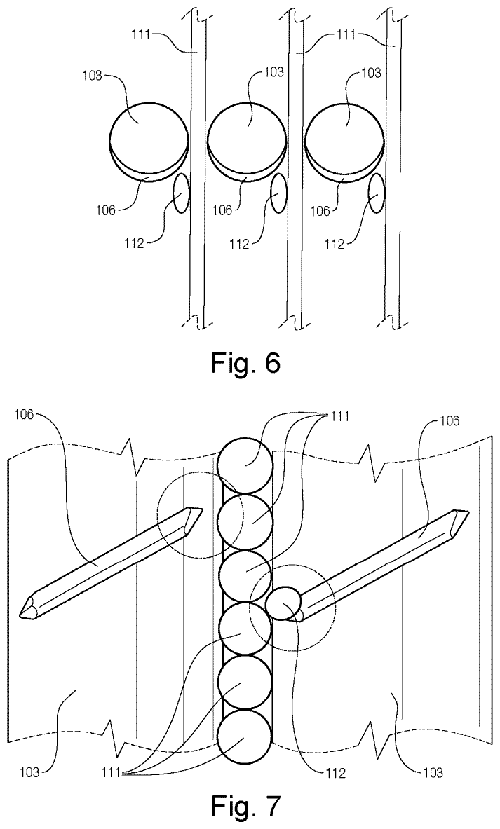

Thus, and according to FIGS. 2 to 7, the comb for the treatment of pediculosis of the present invention is indicated by the general reference 101 and comprises a grip handle 102 comprised of two joined plates and from which a plurality of teeth 103 project, being that said teeth 103 have a proximal end 104 retained in said handle 102 and a free distal end 105 which together define a leading edge with a slightly curved development that first comes into contact with a person's hair and then passes through it and allow the process of removing nits and/or lice.

In accordance with the present invention, each of said teeth comprises a rough means for the retention and removal of adhering material, nits and/or lice present in the hair, defined by a plurality of independent valleys, channels, slots or grooves 106 and separated from each other, each of said grooves 106 being made in a peripheral portion of each tooth 103 so as to extend, from a north pole 107 of the tooth and circumferentially around the tooth, towards both sides of the tooth up to the vicinity of the equator 108 of the tooth or needle cross-section, but without reaching equator 108 at any time in order to avoid the presence of the groove in the area where it scrapes the hair as occurs with the conventional combs of the prior art. In other words, the slot, notch or groove of one needle never faces the slot of an adjacent needle and the gap between needles is always defined by the slot-free needle diameters.

By way of example but not being a limitation for the present invention, said grooves 106 can be made by laser and are arranged or practiced in an inclined manner with respect to the longitudinal geometric axis of each tooth forming an angle of between 40° and 50°, being more preferably 45°. Likewise, said grooves 106 have a depth 113 of between 0.1 mm and 0.3 mm, preferably being 0.2 mm. On the other hand, said grooves 106 are separated from each other by a distance 114 of between 1.0 mm and 3 mm. As can be seen in FIGS. 3, 6 and 7, said grooves 106 of each tooth 103 are arranged in line or opposite to the adjacent grooves 106 of the immediately adjacent tooth 103. In this way, grooves 106 work together to leading nits and/or lice on both sides, significantly improving efficiency in their removal during treatment.

It is once again clarified that said measures are merely exemplary and that they can be modified and adapted to further improve the effectiveness of the comb of the invention. Likewise, by means of the process of engraving the grooves by laser, there is the previously mentioned advantage that the grooves face each other, which cannot be achieved in normal wire scoring processes since the needles or teeth that are later cut, end with the ridges or contiguous needle channels in different positions. On the other hand, it is extremely important to note that the groove 106 does not reach the equator 108 of the section of the needle 103, far from it, but approaches up to a distance or height 109 from it of at least 0.1 mm, being that said distance 109 corresponds according to a tooth or needle diameter of between 0.05 mm to 0.15 mm. Of course, this distance 109 will depend on the diameter chosen for each tooth or needle 103.

With a slot 106 of limited depth, preferably constant along the entire length of the groove except at its ends, and accompanying the periphery of the needle, the resistant section of the needle is not affected, the hair is not damaged, nor is the combability and allows the slots 106 to be closer together, greatly improving removal and cleaning effectiveness. In other words, the two objectives of not reducing combability and achieving the necessary effectiveness in the removal of nits and lice are achieved.

Likewise, the width of each groove 106 can go from a greater width in the central area close to the north pole 107 and decrease towards the equator 108. It is clarified that the shape of each groove 106 can vary without any inconvenience as long as it is kept within the spirit and scope of the object of the present invention.

According to another of the objectives of the present invention, the comb 101 comprises an operative face 110 and an inoperative face opposite to this first one, wherein on said operative face 110, said teeth 103 present said plurality of grooves 106 partially along of its entire length; while on said inoperative face, said teeth 103 are smooth or have no grooves along their entire length. On the other hand, it is highlighted that said teeth 103 are separated from each other by a distance of between 0.05 mm and 0.1 mm. This distance allows the passage of the hair 111 without causing a decrease in combability and at the same time allows the removal of nits and/or lice 112 through the grooves 106.

In this way, the comb for the treatment of pediculosis of the present invention is constituted and constructed, which presents a plurality of grooves arranged in an inclined manner with respect to the geometric axis of the needle, and in turn project from one pole north towards the equator of the section, all this on the peripheral surface of each needle without reducing the effective diameter of each needle or tooth and consequently without altering the effective distance between teeth. It is clarified that, when mentioning “tooth” or “needle”, reference is being made to the same constructive part of the comb.

Thus, the invention provides a comb where:
The fluting goes from smallest to largest from the diameter near the equator of the section where the hair rubs towards the north pole;
The combability of either side is not affected since the diameter is free of engraving;
The rib can be much deeper than before, since it does not affect the hairstyle;
Ribbing is more aggressive since it is done with a laser;
Different grooved figures can be made that provide greater aggressiveness for the nit;
As the nit is exceeding the diameter of the hair, it is well attacked by the ribbing.

Thus, there is a new comb for the treatment of pediculosis that consists of a comb that now does not use ridges in the form of “helical or circular scratches” but rather independent and spaced grooves, all this on a single operative face of the comb, since the other side is free of grooves or is completely smooth.

Also, helical scoring or grooving that was made in the prior art with a compression tool, such as a saw or grinder, is preferably no longer made in this way in accordance with the invention. The grooves of the present invention are engraved by means of a laser process, and laser engraving can be carried out on the finished comb or at least on the welded comb.

In effect, the grooved reeds of the prior art required, before being assembled, that the needles be already slotted or grooved, either circumferentially or helically, after which the needles were cut to the necessary length, arranged one next to the other, with the appropriate separation, and fixed, for example welded, together with the handle. There was then no control over the coincidence or not of the slots between adjacent teeth or over the beginning of each line of slots.

This translates into conventional combs that have, except for Levandosvky, continuous helices on the steel cylinder that protrude from the diameter to leading and break the nit. But since they are continuous, the part of this roughness in contact with the hair significantly worsens combability, and combability is extremely important in the treatment of pediculosis. That is, the rougher, the more effective but more difficult to comb. Although, in the cited case of Levandosvky, the aim is to improve combability, this is not fully achieved since its effectiveness is lost due to the fact that the roughness does not cover the area where the nit would be, resulting in an inefficient comb for the treatment of head lice.

By means of the present invention, all the aforementioned drawbacks are solved thanks to the fact that it provides a comb with “sharp” peripheral grooves that reach the area where the nit is without affecting combability.

According to another aspect of the invention, a method of manufacturing a comb for the treatment of pediculosis is provided, such as the one illustrated and described above, where the method comprises the steps of cutting a plurality of segments from a wire to shaping each of the teeth of the comb, fixing said teeth to a handle arranging one adjacent to the other, leaving an interdental distance between the teeth, subjecting at least one side of the comb to laser engraving to make a plurality of ridges or grooves in the teeth on that side of the comb.

According to the method of the invention, said grooves are made in a peripheral portion of each tooth so as to project from a north pole and circumferentially towards both sides up to the vicinity of the equator of the section of the tooth. And more particularly, said grooves are made to a constant peripheral depth except at the ends of the groove.

The grooves can be engraved on one or both sides of the comb. On the other hand, unlike the prior art where the helical or circular groove occupies the space occupied by the hair, that is, between two teeth or needles, now it occupies the space immediately following the diameter. In this way it does not affect the hair, but it does affect the nit that occupies a larger space. This occurs because when moving away from the vertical between the laser and the steel, the focal length changes and also the geometry does not allow the wire to be affected in its entire circumference. In other words, it becomes very aggressive for the nit and not aggressive for the hair, thus allowing the removal of nits and/or lice without reducing combability.

It is highlighted that the shape and functionality of the handle, union between parts, shape of retention of the teeth 103, distal ends or tips of the teeth or needles 103, are well known in the field of art and that, in turn, by not forming part of the object of the present invention, they will not be described in detail, it being understood that the novel features of the present invention can be adapted and used with any combination of free tips of the needles or teeth, as well as any handle or comb format, as long as it does not depart from the spirit and scope of the object of the present invention.

1. A comb for the treatment of pediculosis comprising:
a grip handle,
a plurality of teeth projecting from the handle, said teeth having a proximal end retained in said handle and a free distal end which together define a leading edge defined to first come into contact with a person's hair and to pass through the hair and allow the process of removing nits and/or lice, wherein said comb is characterized in that:
each of said teeth comprises a roughness defined by a plurality of grooves independent to each other and separated from each other, each of said grooves being made in a peripheral portion of each tooth so as to extend from a north pole of a cross-section of the tooth and circumferentially around the tooth towards both sides of the tooth up to the vicinity of the equator of the tooth cross-section.

2. The comb according to claim 1, comprising an operative face and an inoperative face, wherein:
on said operative face, said teeth have said plurality of grooves partially along their entire length; while
on said inoperative face, said teeth are smooth without grooves along their entire length.

3. The comb according to claim 2, wherein said grooves are inclined with respect to a longitudinal axis of each tooth forming an angle of between 40° and 50°.

4. The comb according to claim 3, wherein said angle is 45°.

5. The comb according to claim 1, wherein said grooves are made by laser.

6. The comb according to claim 5, wherein said grooves have a depth of between 0.1 mm to 0.3 mm.

7. The comb according to claim 5, wherein said grooves have a depth of 0.2 mm.

8. The comb according to claim 1, wherein said grooves are separated from each other by a distance of between 1.5 mm and 3 mm.

9. The comb according to claim 1, wherein said grooves of each tooth are arranged in line with and adjacent to the grooves of the immediately adjacent tooth.

10. The comb according to claim 1, wherein each groove extends circumferentially from the north pole to a distance or height of at least 0.1 mm from the equator of the section.

11. The comb according to claim 1, wherein said teeth are separated from each other by a distance of between 0.05 mm and 0.1 mm.

12. A method of manufacturing a comb for the treatment of pediculosis of the type that comprises a grip handle from which a plurality of teeth project, said teeth having a proximal end retained in said handle and a free distal end which together define a leading edge defined to first come into contact with a person's hair and to pass through the hair and allow the process of removing nits and/or lice, the method comprising the steps of:
cutting, from a wire, a plurality of segments to form each of the teeth of the comb,
fixing said teeth to the grip handle, arranging one tooth adjacent to the other, leaving an interdental distance between teeth,
subjecting at least one side of the comb to laser engraving to make a plurality of grooves in the teeth only on said side of the comb.

13. A method according to claim 12, wherein said grooves are made in a peripheral portion of each tooth so as to project from a north pole of the tooth cross-section and circumferentially around the tooth towards both sides up to the vicinity of the equator of the cross-section of the tooth.

14. The method according to claim 13, wherein said grooves are made to a constant peripheral depth except at the groove end.
